(19) United States
(12) Patent Application Publication
(10) Pub. No.: US 2008/0023004 A1
(43) Pub. Date: Jan. 31, 2008

(54) Control valve

(75) Inventor: Sen-Yu Wu, Taichung Hsien (TW)

Correspondence Address: BRUCE H. TROXELL;SUITE 1404, 5205 LEESBURG PIKE, FALLS CHURCH, VA, 22041, US

(73) Assignee: Seven Universe Industrial Co., Ltd.

(21) Appl. No.: 11/486,207

(22) Filed: Jul. 14, 2006

(51) Int. Cl. A61M 16/00; A62B 7/00

(52) U.S. Cl. 128/204.21

(57) ABSTRACT

The invention is providing an improved control valve, in the connecting mechanism of a gas container and a pressure regulator consisting a valve body, set up with a hollow receiving room at one end and penetrating with a 1st flow field linked with a receiving room, which is set up with a control mouth at the joint of the receiving room and 1st flow field; a fitting is set up in that receiving room and a 2nd flow field is linked with the control mouth in a expected distance; a magnet is set up in an interval between the fitting and the control mouth; a slippery metal ball is absorbed by the magnetism of the magnet, which outer diameter is larger than the bore of the control mouth; wherein, when the fluid flow in the 1st and 2nd flow field increases so that the attraction to the ball is stronger than the magnetism attraction of the magnet or the magnet is demagnetized because of being heated up to an expected temperature, the ball will fall off and driven by the fluid to shut the control mouth so as to break the 1st and 2nd flow field and further generate the effect of super fluid and overheat shutting fluid.

### BACKGROUND OF THE INVENTION

1. Field of the Invention

The invention relates to a gas device and in detail to a control valve having the effect of super fluid and overheat shutting fluid.

2. Description of the Related Art

According to the connecting mechanism of the conventional gas container and pressure regulator is connected between a gas container (like gas bucket) and a burning device (like gas stove and grill stove), mainly conveyed to be burning in a burning device through a pipe by controlling the gas in expected pressure and flow rate. Its inner part is mostly set up with a control valve to avoid unsafe condition due to the gas super fluid. The control valve of the connecting mechanism of the conventional gas container and pressure regulator has patterns like the ball bearing super fluid control valve and the magnetic super fluid control valve, etc. The composition of a ball bearing super fluid control valve is mainly set up with a fillister in front of the control mouth inside the valve body, which there is a ball bearing in the fillister so that when the gas super fluid occurs in the valve body, the ball bearing will be forced to resist and withhold and generate the effect of shutting the control mouth to shut the super fluid. And the magnetic super fluid control valve mainly changes the above-mentioned ball bearing into an iron piece which will be attracted by the magnetism of the front magnet so that when the super fluid occurs in the valve body, the iron piece will fall off from the magnet due to the force of the gas fluid stronger than the magnetism of the front magnet and shut the control mouth. The structure of the above-mentioned ball bearing super fluid control valve has its directional problem so it will easily cause the inconvenience of fabrication. And the magnetism of the magnetic super fluid control valve will be easily affected by the external factors (such as temperature, electric current or outer magnetic force, etc.) and lead to the phenomenon of magnetization or demagnetization which its practicability needs to be promoted.

In order to solve the defects of the above-mentioned conventional control valve, some set up a strip in the center of the gas flow field to control of the control mouth is freely flowing. Both sides of the strip are formed a gas passage with the gas flow field. When the super fluid changes, the strip will be influenced and present a curve because the super fluid force is stronger than the rigidity and flexibility itself and further shut the control mouth. Although this design can truly solve the conventional the defects of the bearing or the magnetic super fluid control valve, due to the strip is set up in between the gas flow field, when the gas flows, it will easily strike against the strip and cause it vibrate by the turbulence in use and generate noises, furthermore, when the super fluid flows, the strip will easily in a critical status of shutting or not shutting (shut the control mouth). (When the strip bends and becomes deformed, its elastic force will keep stronger and become unstable status of shutting or not shutting when the super fluid force overcomes the critical point of the strip's elasticity).

### SUMMARY OF THE INVENTION

The main purpose of the invention is to provide with a control valve to solve the above-mentioned defects with better practicality of convenient assembling and exact action.

Another purpose of the invention is to provide with an improved control valve which has effect of shutting the gas flow field when overheat happens and has better safety.

Therefore, in order to achieve the above-mentioned purpose, the invention is to provide with an improved control valve which is located in the joint mechanism of a gas container and a pressure regulator, including a valve body, a hollow receiving room set up at one end, a 1st flow field linked with the receiving room set up at the other end, wherein a control mouth is set up at the joint of the receiving room and 1st flow field; a fitting is et up in the receiving room and has and a 2nd flow field is linked with the control mouth in a expected distance; a magnet is set up between the fitting and the control mouth; a slippery metal ball is absorbed by the magnetism of the magnet, which outer diameter is larger than the bore of the control mouth; wherein, when the fluid flow in the 1st and 2nd flow field increases so that the attraction to the ball is stronger than the magnetism attraction of the magnet or the magnet is demagnetized because of being heated up to an expected temperature, the ball will fall off and driven by the fluid to shut the control mouth so as to break the 1st and 2nd flow field.

### BRIEF DESCRIPTION OF THE DRAWINGS

Shown as follows, here are several better practical examples of the invention and in accommodation with figures to further describe in details as follows:

FIG. 1 is the assembling schematic view of the first better practical examples of the invention.

FIG. 2 is the motion schematic view of the first better practical examples of the invention which shows the status of the ball is stuck at the control mouth.

FIG. 3 is the assembling schematic view of the second better practical examples of the invention.

FIG. 4 is the motion schematic view of the second better practical examples of the invention which shows the status of the ball is stuck at the control mouth.

FIG. 5 is the assembling schematic view of the third better practical examples.

### DETAILED DESCRIPTION OF THE INVENTION

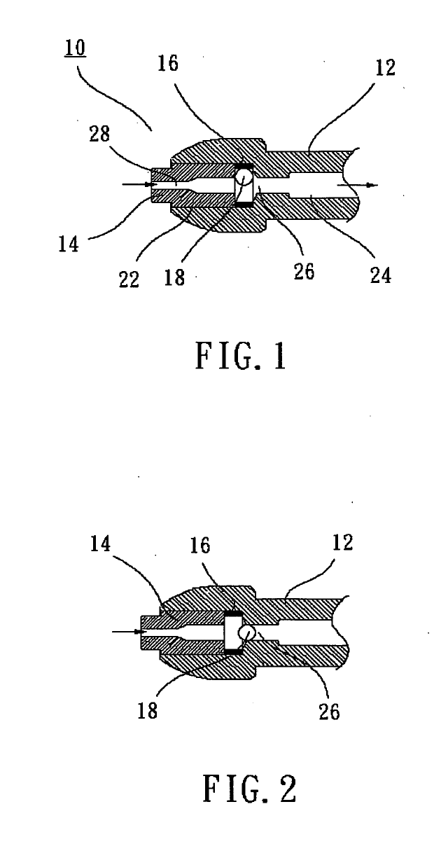

As show in FIG. 1, the Control Valve 10 of the better practical examples of the invention is located in the joint mechanism of a gas container and a pressure regulator (not show in the figure) consisting by a valve body 12, a fitting 14, a magnet 16 and a metal ball 18.

At one end of the valve body 12 there is set up with a hollow receiving room 22; at the other end there is set up with a first flow field 24 linked with the receiving room 22, wherein is set up with a control mouth 26 at the joint between the receiving room 22 and first flow field 24.

The fitting 14 is set up in the receiving room 22 and a second flow field 28 is linked with the control mouth 26 in an expected distance.

The magnet 16 is set up in an interval between the fitting 14 and the control mouth 26. The slippery metal ball 18 is absorbed by the magnetism of the magnet 16, which outer diameter is larger than the bore of the control mouth 26.

Thus, the fluid flow in the first and second flow field 24 and 28 in the control valve 10 can be provided for the gas (or fuel) flowing within. When there is no fluid (gas) flowing in the control valve 10 or flowing in standard, the ball 18 is absorbed by the magnetism of the magnet 16. The first and second flow field 24 and 28 keep flowing freely. When the fluid flow suddenly increases (super fluid) and the absorbing force generated overcome the magnetic absorbing force generated from the magnet 16 against the ball 18, then the ball 18 can be absorbed and stick the control mouth 26 as shown in FIG. 2, so as to make the control valve 10 shut to keep the safety when in use.

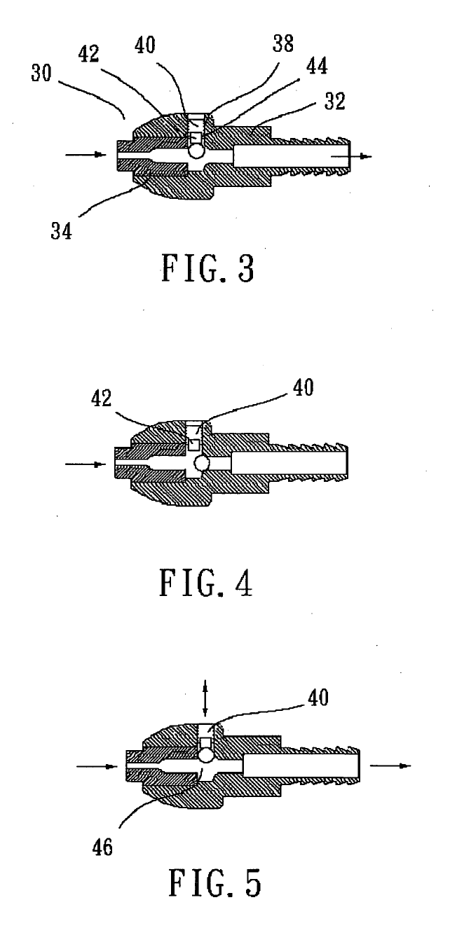

As shown in FIG. 3, the control valve 30 is anther better practical example of the invention. Its structure is the same with the control valve 10 in all. Its differences are: the position of valve body 32 relatively to the end of fitting 34 and the interval of control mouth 36 is further set up with a receiving holes 38, there is a screw holes, a adjusting component 40, there screw bolts, which screwed in the receiving holes 38. The magnet 42 is in a block shape fixed in the socket 44 inside the adjusting component 40. In this way, the control valve 30 not only has the conventional effect of super fluid shutting. As shown in FIG. 4, furthermore, the flow valve of the super fluid shutting according to the requirement of different gas device can be set up. That is, as shown in FIG. 5, if the adjusting component 40 is regulated to the position farther from the center of the gas flow field 46, a motion and effect of generating super fluid shutting needs higher flow. On the contrary, if the adjusting component 40 is regulated to the position nearer the gas flow field 46, the lower flow of super fluid can generate the safety effect of super fluid shutting.

Besides, the magnet of the control valve of the invention can also used as a conventional temperature memory magnet that the magnetism can be restored so that when the temperature of the control valve rises to the set value due to unusual condition, the magnet shall be then demagnetized so that the control valve can be shut even under a gas flow condition to keep safe. However, after the control valve is shut to decrease the temperature under the set value, the magnetism of the magnet will restore so as to form a overheat safety device with an automatic restoration. Certainly, the magnet can be a conventional non-restored temperature memory magnet so that after the control valve is shut due to overheat, the magnet is permanently demagnetized and the magnetism will not restore so as to form a overheat safety device with function of temperature fuse.

What is claimed is:

1. An improved control valve in the connecting mechanism of a gas container and a pressure regulator comprising:
a valve body set up a hollow receiving room at one end and a first flow field linked with a receiving room at the other end, wherein there is set up with a control mouth at the joint of the receiving room and first flow field;
a fitting set up in the receiving room with the control mouth in a expected distance and control mouth, with a second flow field linked with the control mouth;
a magnet set up between the fitting and the control mouth; and
a metal ball a slippery metal ball is absorbed by the magnetism of the magnet, which outer diameter is larger than the bore of the control mouth;
wherein, when the fluid flow in the first and second flow field increases so that the attraction to the ball is stronger than the magnetism attraction of the magnet or the magnet is demagnetized because of being heated up to an expected temperature, the ball will fall off and driven by the fluid to shut the control mouth so as to break the first and second flow field.

2. The improved control valve as defined in claim 1, wherein the magnet is a ring shape and the ball is absorbed ant attached inside the magnet by the magnetism.

3. The improved control valve as defined in claim 1, wherein the magnet is a block shape.

4. The improved control valve as defined in claim 1, wherein the valve body on the position relative to the magnet is further set up a receiving hole, a adjusting component is removably set up in the receiving holes, where the magnet is fixed inside the adjusting component and absorbed and attached the ball with magnetism.

5. The improved control valve as defined in claim 4, wherein the adjusting component is made up of metal material and the magnet is absorbed and attached with magnetism in the inner part.

6. The improved control valve as defined in claim 5, wherein the receiving holes are screw holes and the adjusting components are screw bolts.

7. The improved control valve as defined in claim 4, wherein inside the adjusting component there is set up with a socket and the magnet is set up in the socket.

8. The improved control valve as defined in claim 1, wherein the magnet is a conventional temperature memory magnet with restorable magnetism.

9. The improved control valve as defined in claim 1, wherein the magnet is a conventional temperature memory magnet with non-restorable magnetism.
